(19) United States
(12) Patent Application Publication
(10) Pub. No.: US 2003/0216407 A1
(43) Pub. Date: Nov. 20, 2003

(54) Use of PDE5 inhibitors in the treatment of scarring

(75) Inventors: Richard Philip Butt, Sandwich (GB); Stephen Charles Phillips, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/350,933

(22) Filed: Jan. 24, 2003

Related U.S. Application Data
(60) Provisional application No. 60/362,425, filed on Mar. 6, 2002.

(30) Foreign Application Priority Data
Jan. 31, 2002 (GB) 0202254.9

(51) Int. Cl. A61K 31/519

(52) U.S. Cl. 514/252.16

(57) ABSTRACT

This invention relates to the use of selective cyclic guanosine 3′,5′-monophosphate type five (cGMP PDE5) inhibitors (hereinafter PDE5 inhibitors), including in particular the compound sildenafil, for the treatment of or prevention of scarring or fibrosis in tissue.

The present invention relates to the use of cyclic guanosine 3′,5′-monophosphate type five cGMP PDE 5 inhibitors (hereinafter PDE 5 inhibitors), including in particular the compound sildenafil, for the reduction of or prevention of scarring and/or fibrosis.

In accordance with the present invention, examples of disease associated with scarring and/or fibrosis include (but are not necessarily limited to): lung fibrosis, atherosclerosis, cardiovascular disease, dermal and corneal scarring and/or fibrosis following infection, trauma, surgery or thermal injury, scleroderma and other connective tissue disorders, fibrosis of the heart, chronic obstructive pulmonary disease, muscle fibrosis, kidney fibrosis, chronic dermal ulceration and lipdermatosclerosis, lung fibrosis or any origin), post-surgical and idiophatic adhesions, inflammatory conditions of the skin (including lichen and associated conditions), ageing and all ageing associated degenerative disorders (including ageing of the skin), liver fibrosis or any etiology (including viral and non-viral hepatitis, liver cirrhosis, chronic pancreatitis, chronic thyroiditis, calcinosis (of any origin), conditions whose pathogenesis is related to the deposition/remodelling of a connective matrix (including cancer).

The present invention relates to the use of certain compounds in the treatment of such disease states.

The incidence of some diseases associated with scarring and/or fibrosis is a significant drain on resources in both developing and developed nations. The costs for both national and international public health programs attempting to deal with the consequences of these diseases are substantial. It would therefore be desirable to provide a means for treating or reducing the effects of diseases associated with scarring and/or fibrosis.

The progression of certain diseases associated with scarring and/or fibrosis such as atherosclerosis may involve the accumulation/proliferation of smooth muscle cells )SMCs) which elaborate extracellular matrix micromolecules which are largely collagenous in nature. The progression of atherosclerosis from thrombosis to myocardial infarcation (MI) can lead to tissue injury, which may result in both scar tissue turnover and fibrous tissue formation. Although the process of normal wound repair after tissue injury results in the proliferation of fibroblast cells, the differentiation of fibroblasts into myofibroblasts can mark an early event in the development of tissue fibrosis. The prolonged presence of myofibroblasts at an infarct site may also be likely to produce an inbalance in extracellular matrix proteins and proteases, which may exacerbate hypertrophic scars and wound formation.

It would be desirable to provide compounds for the treatment of diseases associated with scarring and/or fibrosis which are capable of treating or at least ameliorating these disease states.

EPO930069 discloses compositions for the reduction of scarring. Amongst these compositions are phosphodiesterase inhibitors which are said to reduce wound scarring. However, the phosphodieterase inhibitors described in that document are broad-spectrum inhibitors and are not specific for PDE 5. As such, the inhibitors of this patent may be disadvantageous in that they do not have the same therapeutic efficiency as the compounds of the present invention.

Redondo et al (British Journal of Pharmacology 1998, 124, 1455-1462) describe a study for the effect of atrial natriurectic peptide (ANP) and cyclic GMP phosphodiesterase inhibition on collagen synthesis by adult cardiac fibroblasts. Two major subclauses of natriurectic peptide receptors have been indentified of which the NPR-C type is the most dominant, accounting for 70% of the natriurectic peptide receptor population in cariac fibroblasts. The authors found that the PDE inhibitor, zaprinast had no effect on its own in regulating cardiac fibroblast proliferation. Similarly, ANP did not on its own regulate cardiac fibroblast proliferation. However, the combination of ANP and zaprinast did produce a concentration—dependent inhibition of thymidine incorporation over a limited concentration range and the authors used this as an indirect assay of DNA synthesis. The results could not be reproduced with C-ANF4-32 (a NPR-C specific analogue) in combination with Zaprinast. Further doubt is cast over this study by the authors statement that zaprinast is a specific PDE5 inhibitor when in fact it is documented elsewhere in the literature that zaprinast acts as a non-specific PDE inhibitor (see, for example, McMahon et al) (doc 18) and Kukoretz et al (doc 3). It is also known that it is five fold more potent against PDE6 than against PDE5.

Duncan et al (The FASEB Journal) discloses in vitro studies on normal rat kidney in which it was found that connective tissue growth factor mediates transforming growth factor beta (TGF-β)-induced fibroblast collagen synthesis and that in vivo blockade of CTGF synthesis or action reduces TGF-β induced granulation tissue formation by inhibiting both collagen synthesis and fibroblast accumulation. cAMP also inhibited collagen synthesis induced by CTGF itself whereas cGMP was reported to have no effect. This paper contradicts the hypothesis by Redondo et al that PDE5 can inhibit collagen production. Thus the role of cGMP in scarring is unclear from the art.

The process of wound repair following disruption of tissue homeostatis involves a cascade of coordinately linked overlapping phases which includes: inflammation, granulation tissue formation, extracellular matrix deposition and assembly, and termation. Peptide factors are involved in the process in various ways and control platelet function, leukotaxis, cytokine synthesis, and agiongenesis as well as directing the progression of fibroblast phenotypes that ultimately results in the formation of premature scare tissue. The peptide factors exercise control over these processes by regulating the ability of fibroblasts to proliferate and to quantitatively and qualitatively change their extracellular matrix component production profiles. One of the primary regulatory factors known to be involved in initiating the wound healing cascade is TGF-β.

There is some suggestion in the literature that nitric oxide improves the rate of wound healing. It is also known that cGMP PDE5 inhibitors increase intracellular concentrations of nitric oxide derived cGMP, thereby enhancing the effect of nitric oxide, which is responsible for the efficacy of sildenafil in the treatment of male erectile dysfunction.

Without wishing to be bound by theory, it is believed that the antiscarring effect is linked to specific PDE 5 inhibition at an appropriate stage in the wound-healing cycle. This may occur in conjunction with an appropriate signal such as NO-mediated smooth muscle relaxation. Other factors may also be involved.

Surprisingly, we have thus found that administration of a PDE 5 inhibitor to a healing wound can result in a reduced incidence of scar tissue formation.

We have found from in vivo observations in the fibrosis of heart tissue that there is excessive protein PDE5 expression relative to normal heart tissue. We have also determined that the PDE5 is present in a sub-population of fibroblasts known as myofibroblasts. Increased PDE5 expression in these cells may therefore be involved in the pathophysiology that leads to tissue fibrosis. The mechanisms leading to fibrosis in all tissues is thought it to be similar and thus fibrosis occurring in the liver, kidney, lungs, spinal cord, and skin will proceed similarly. In accordance with the present invention the fibrotic conditions of all these tissue types (and many others) may be alleviated by PDE5 inhibition thus leading to a significant therapeutic benefit.

Although non-selective PDE inhibition (as exemplified by Redondo et al in a study of zaprinast, and data using pentoxyfilline (a weak and non-selective PDE inhibitor), has suggested that these agents may behave as antifibrotic agents there has not been any recognition in the prior art that a treatment to prevent or reduce scarring could be based on selective PDE5 inhibition. Indeed, there seems to be a conflict of opinion in the prior art regarding the role of cGMP (and hence the role of PDE5) in scar formation.

According to a first aspect of the present invention, there is provided a method for reducing scarring and/or treating fibrosis in a patient which comprises treating the patient with an effective amount of a cGMP PDE 5 inhibitor or a pharmaceutical composition thereof.

According to a second aspect of the present invention, there is provided the use of cGMP PDE 5 inhibitor for the manufacture of a medicament for reducing scarring and/or treating fibrosis.

According to a third aspect of the present invention, there is provided the use of cGMP PDE 5 inhibitor for reducing scarring and/or treating fibrosis in tissue.

According to a fourth aspect of the present invention there is provided a pharmaceutical pack comprising: a pharmaceutical composition comprising a PDE5 inhibitor, directions relating to the use of the composition for reducing scarring and/or treating fibrosis, and a container.

In an embodiment of each of the above aspects, diseases associated with scarring and/or fibrosis which are capable of treatment in accordance with the invention include: lung fibrosis, atherosclerosis, cardiovascular disease, dermal and corneal scarring and/or fibrosis following infection, trauma, surgery or thermal injury, scleroderma and other connective tissue disorders, fibrosis of the heart, chronic obstructive pulmonary disease, muscle fibrosis, kidney fibrosis, chronic dermal ulceration and lipdermatosclerosis, lung fibrosis or any origin), post-surgical and idiophatic adhesions, inflammatory conditions of the skin (including lichen and associated conditions), ageing and all ageing associated degenerative disorders (including ageing of the skin), liver fibrosis or any etiology (including viral and non-viral hepatitis, liver cirrhosis, chronic pancreatitis, chronic thyroiditis, calcinosis (of any origin), conditions whose pathogenesis is related to the deposition/remodelling of a connective matrix (including cancer).

No therapeutic agent is currently commercially available which improves or prevents the incidence of scarring in tissue by acting selectively on the cGMP PDE5 isoenzyme.

In the context of the present invention, PDE5 inhibitor refers to any compound which is a potent and selective inhibitor of the cGMP PDE5 isoenzyme.

For the purposes of the present invention, the PDE5 inhibitor must demonstrate a selectivity of at least 25 fold, and preferably at least 30 fold, in favour of PDE5 inhibition.

Suitable PDE5 inhibitors for use in the pharmaceutical combinatiions according to the present invention are the cGMP PDE5 inhibitors hereinafter detailed. Particularly preferred for use herein are potent and selective cGMP PDE5 inhibitors.

Suitable cGMP PDE5 inhibitors for the use according to the present invention include:

the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the compounds disclosed in published international application WO95/19978; the compounds disclosed in published international application WO 99/24433 and the compounds disclosed in published international application WO 93/07124.

The pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclosed in EP-A-1092719.

Preferred type V phosphodiesterase inhibitors for the use according to the present invention include:

5-[2-ethoxy-5-(4-methyl-1-piperazinylsulphonyl)phenyl]-1-methyl-3-n-propyl-1,6-dihydro-7H-pyrazolo[4,3-d]pyrimidin-7-one(sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);

5-(2-ethoxy-5-morpholinoacetylphenyl)-1-methyl-3-n-propyl-1,6-dihydro-7H-pyrazolo[4,3-d]pyrimidin-7-one (see EP-A-0526004);

3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);

3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333);

(+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333);

5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8);

5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);

5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);

5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124);

5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132);

(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8;

2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; and

the compound of example 11 of published international application WO93/07124 (EISAI); and

compounds 3 and 14 from Rotella D P, J. Med. Chem., 2000, 43, 1257.

Still other type cGMP PDE5 inhibitors useful in conjunction with the present invention include:4-bromo-5-(pyridylmethylamino)-6-[3-(4-chlorophenyl)-propoxy]-3(2H)pyridazinone; 1-[4-[(1,3-benzodioxol-5-ylmethyl)amino]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3(2H)pyridazinone; I-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

For the avoidance of doubt, the PDE 5 inhibiting compounds referred to above which are described in detail in the referenced published patent specifications mentioned above specifically form a part of this disclosure and represent a part of the inventive subject matter of this application.

The suitability of any particular cGMP PDE5 inhibitor can be readily determined by evaluation of its potency and selectivity using literature methods followed by evaluation of its toxicity, absorption, metabolism, pharmacokinetics, etc in accordance with standard pharmaceutical practice.

Preferably, the cGMP PDE5 inhibitors have an IC50 for PDE5 at less than 100 nanomolar, more preferably, at less than 50 nanomolar, more preferably still at less than 10 nanomolar.

IC50 values for the cGMP PDE5 inhibitors may be determined using established literature methodology, for example as described in EP0463756-B1 and EP0526004-A1.

Preferably the cGMP PDE5 inhibitors used in the invention are selective for the PDE5 enzyme. Preferably they are selective over PDE3, more preferably over PDE3 and PDE4. Preferably, the cGMP PDE5 inhibitors of the invention have a selectivity ratio greater than 25, more preferably greater than 30, and still more preferably greater than 100, over PDE3 and more preferably over PDE3 and PDE4. The best inhibitors show a selectivity of preferably greater than 300, over PDE3 and more preferably over PDE3 and PDE4.

Selectivity ratios may readily be determined by the skilled person. IC50 values for the PDE3 and PDE4 enzyme may be determined using established literature methodology, see S A Ballard et al, Journal of Urology, 1998, vol. 159, pages 2164-2171.

To be effective as a treatment, the compounds of the invention are preferably orally bioavailable. Oral bioavailablity refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailablity.

Dissolution, the solubilisation of the drug by the aqueous contents of the gastro-intestinal tract (GIT), can be predicted from in vitro solubility experiments conducted at appropriate pH to mimic the GIT. Preferably the compounds of the invention have a minimum solubility of 50 mcg/ml. Solubility can be determined by standard procedures known in the art such as described in Adv. Drug Deliv. Rev. 23, 3-25, 1997.

Membrane permeability refers to the passage of the compound through the cells of the GIT. Lipophilicity is a key property in predicting this and is defined by in vitro Log D_{7.4} measurements using organic solvents and buffer. Preferably the compounds of the invention have a Log D_{7.4} of −2 to +4, more preferably −1 to +2. The log D can be determined by standard procedures known in the art such as described in J. Pharm. Pharmacol. 1990, 42:144.

Cell monolayer assays such as caco-2 add substantially to prediction of favourable membrane permeability in the presence of efflux transporters such as p-glycoprotein, so-called caco-2 flux. Preferably, compounds of the invention have a caco-2 flux of greater than 2×10⁻⁶ cms⁻¹, more preferably greater than 5×10⁻⁶ cms⁻¹. The caco flux value can be determined by standard procedures known in the art such as described in J. Pharm. Sci, 1990, 79, 595-600

Metabolic stability addresses the ability of the GIT or the liver to metabolise compounds during the absorption process: the first pass effect. Assay systems such as microsomes, hepatocytes etc are predictive of metabolic liability. Preferably the compounds of the Examples show metabolic stability in the assay system that is commensurate with an hepatic extraction of less then 0.5. Examples of assay systems and data manipulation are described in Curr. Opin. Drug Disc. Devel., 201, 4, 36-44, Drug Met. Disp.,2000, 28, 1518-1523

Because of the interplay of the above processes further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Drug Met. Disp.,2001, 29, 82-87; J. Med Chem, 1997, 40, 827-829, Drug Met. Disp.,1999, 27, 221-226

Preferably the cGMP PDE5 inhibitor is Sildenafil.

The cGMP PDE5 inhibitors can be administered alone but, in human therapy will generally be administered in admixture with a suitable pharmaceutical excipient diluent or carrier selected with regard to the intended route of administration and standard pharmaceutical practice.

For example, the cGMP PDE5 inhibitors can be administered orally, buccally or sublingually in the form of tablets, capsules, ovules, elixirs, solutions or suspensions, which may contain flavouring or colouring agents, for immediate-, delayed-, modified-, or controlled-release applications.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethyl cellulose, hydroxypropylcellulose, sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the cGMP PDE5 inhibitors of the invention may be combined with various sweetening or flavouring agents, colouring matter or dyes, with emulsifying and/or suspending agents and with diluents such as water, ethanol, propylene glycol and glycerin, and combinations thereof.

The cGMP PDE5 inhibitors can also be administered parenterally, for example, intravenously, intra-arterially, intraperitoneally, intramuscularly or subcutaneously, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The following dosage levels and other dosage levels herein are for the average human subject having a weight range of about 65 to 70 kg. The skilled person will readily be able to determine the dosage levels required for a subject whose weight falls outside this range, such as children and the elderly.

The dosage of cGMP PDE5 inhibitor in such formulations will depend on its potency, but can be expected to be in the range of from 1 to 500 mg for administration up to three times a day. For oral and parenteral administration to human patients, the daily dosage level of the cGMP PDE5 inhibitor will usually be from 5 to 500 mg (in single or divided doses). In the case of sildenafil, a preferred dose is in the range 10 to 100 mg (e.g. 10, 25, 50 and 100 mg) which can be administered once, twice or three times a day (preferably once). However the precise dose will be as determined by the prescribing physician and will depend on the age and weight of the patient and severity of the symptoms.

Thus, for example, tablets or capsules of the cGMP PDE5 inhibitor may contain from 5 to 250 mg (e.g. 10 to 100 mg) of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The cGMP PDE5 inhibitors can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane or 1,1,1,2,3,3,3-heptafluoropropane, carbon dioxide or other suitable gas. In the case of a pressurised aerosol, the dosage unit may be determined by providing a valve to deliver a metered amount. The pressurised container, pump, spray or nebuliser may contain a solution or suspension of the cGMP PDE5 inhibitor, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the cGMP PDE5 inhibitor and a suitable powder base such as lactose or starch.

Aerosol or dry powder formulations are preferably arranged so that each metered dose or “puff” contains from 1 to 50 mg of the cGMP PDE5 inhibitor, for delivery to the patient. The overall daily dose with an aerosol will be in the range of from 1 to 50 mg which may be administered in a single dose or, more usually, in divided doses throughout the day.

Alternatively, the cGMP PDE5 inhibitors can be administered in the form of a suppository or pessary.

The cGMP PDE5 inhibitor may be applied topically in the form of a gel, hydrogel, lotion, solution, cream, ointment or dusting powder. The cGMP PDE5 inhibitors may also be dermally or transdermally administered, for example, by the use of a skin patch.

For application topically to the skin, the cGMP PDE5 inhibitors can be formulated as a suitable ointment containing the inhibitor suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The cGMP PDE5 inhibitors may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

Generally, in humans, oral administration of the cGMP PDE5 inhibitors is the preferred route, being the most convenient. In circumstances where the recipient suffers from a swallowing disorder or from impairment of drug absorption after oral administration, the drug may be administered parenterally, sublingually or buccally.

The cGMP PDE5 inhibitors of the invention can also be administered in combination with one or more of the following:

i) α-Adrenergic receptor antagonist compounds also known as α-adrenoceptors or α-receptors or α-blockers. Suitable compounds for use herein include: the α-adrenergic receptors as described in PCT application WO99/30697 published on 14th Jun. 1998, the disclosures of which relating to α-adrenergic receptors are incorporated herein by reference and include, selective α₁-adrenoceptors or α₂-adrenoceptors and non-selective adrenoceptors, suitable α₁-adrenoceptors include: phentolamine, phentolamine mesylate, trazodone, alfuzosin, indoramin, naftopidil, tamsulosin, dapiprazole, phenoxybenzamine, idazoxan, efaraxan, yohimbine, rauwolfa alkaloids, Recordati 15/2739, SNAP 1069, SNAP 5089, RS17053, SL 89.0591, doxazosin, terazosin, abanoquil and prazosin; α₂-blockers from U.S. Pat. No. 6,037,346 [14th Mar. 2000] dibenarnine, tolazoline, trimazosin and dibenarnine; α-adrenergic receptors as described in U.S. Pat. Nos.: 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptors include: clonidine, papaverine, papaverine hydrochloride, optionally in the presence of a cariotonic agent such as pirxamine;

ii) NO-donor (NO-agonist) compounds. Suitable NO-donor compounds for use herein include organic nitrates, such as mono- di or tri-nitrates or organic nitrate esters including glyceryl brinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso- N-acetyl penicilliamine (SNAP) S-nitroso-N-glutathione (SNO-GLU), N-hydroxy-L-arginine, amylnitrate, linsidomine, linsidomine chlorohydrate, (SIN-1) S-nitroso-N-cysteine, diazenium diolates,(NONOates), 1,5-pentanedinitrate, L-arginene, ginseng, zizphi fructus, molsidomine, Re-2047, nitrosylated maxisylyte derivatives such as NMI-678-11 and NMI-937 as described in published PCT application WO 0012075;

iii) Vasodilator agents. Suitable vasodilator agents for use herein include nimodepine, pinacidil, cyclandelate, isoxsuprine, chloroprumazine, halo peridol, Rec 15/2739, trazodone, pentoxifylline;

iv) Thromboxane A2 agonists;

v) Substrates for NO-synthase, such as L-arginine;

vi) Calcium channel blockers such as amlodipine;

vii) Steroidal or non-steroidal anti-inflammatory agents;

viii) Matrix metalloprotease inhibitors (MMP), particularly MMP-3, MMP-12 and MMP-13;

ix) Urokinase type plasminogen activator inhibitors (uPA);

x) PCP inhibitors; and

xi) PDE4 inhibitors.

Particularly preferred agents for use in combination with the PDE5 inhibitors of the invention for treating wounds include: PCP inhibitors such as those of WO 01/47901, GB 0108097.7, PCT/IB01/02360 and GB 0108102.5.

Preferably the MMP inhibitor is a MMP-3 and/or MMP-13 inhibitor such as those specifically and generically disclosed in WO99/35124, EP 931788, WO99/29667 or WO00/74681. Especially preferred MMP inhibitors are those of the Examples of WO99/35124, EP 931788, WO99/29667 and WO00/74681.

Preferably the uPA inhibitor is selected from those specicially and generically disclosed in WO99/20608, EP 1044967 or WO00/05214. Especially preferred uPA inhibitors are those of the Examples of WO99/20608, EP 1044967 and WO00/05214.

It is to be appreciated that all references herein to treatment include curative, palliative and prophylactic treatment.

The utility of the present invention is illustrated by the following figures in which:

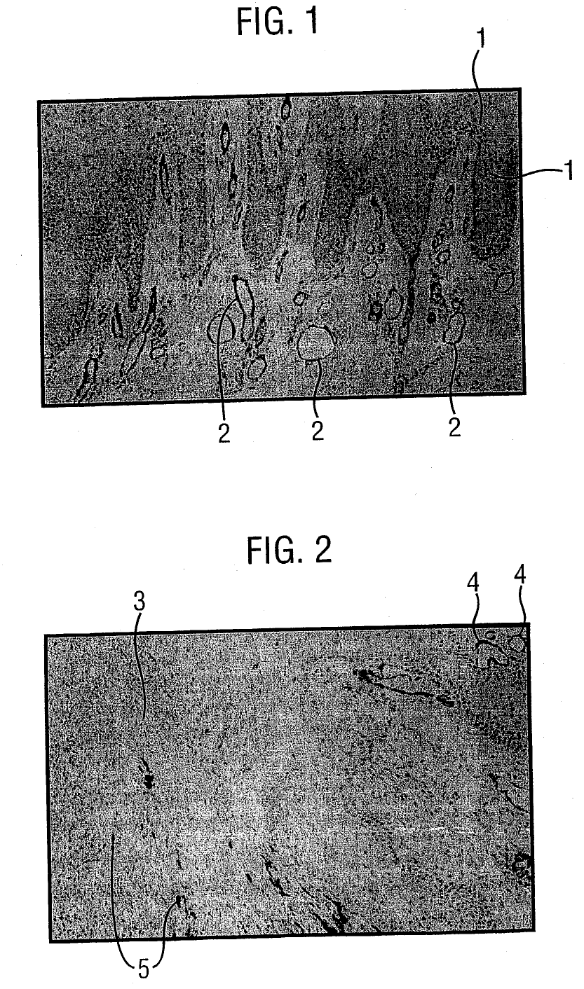

FIG. 1 is a photomicrograph of a paraffin section of skin at 10×magnification;

FIG. 2 is a photomicrograph of a paraffin section of skin at 20×magnification;

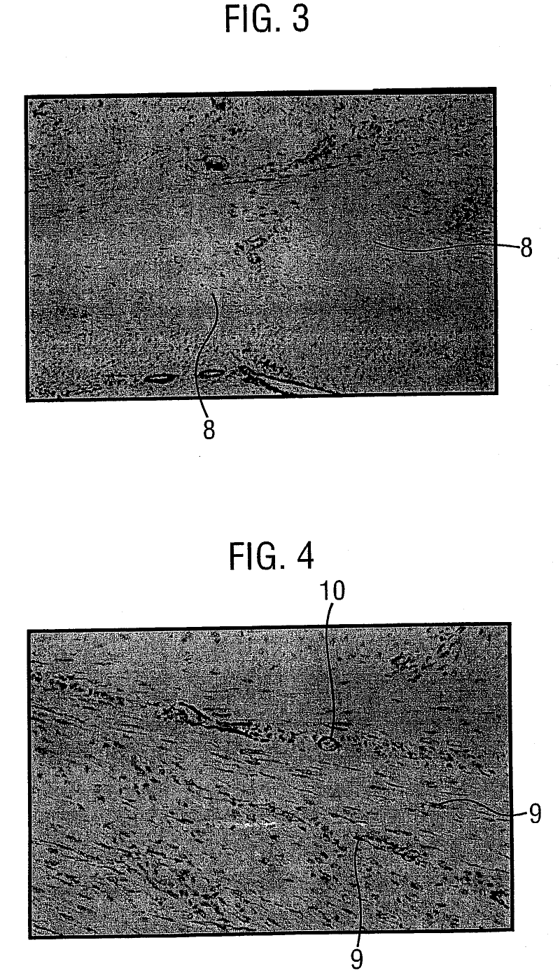

FIG. 3 is a photomicrograph of a paraffin section of skin at 20×magnification;

FIG. 4 is a photomicrograph of a paraffin section of skin at 40×magnification;

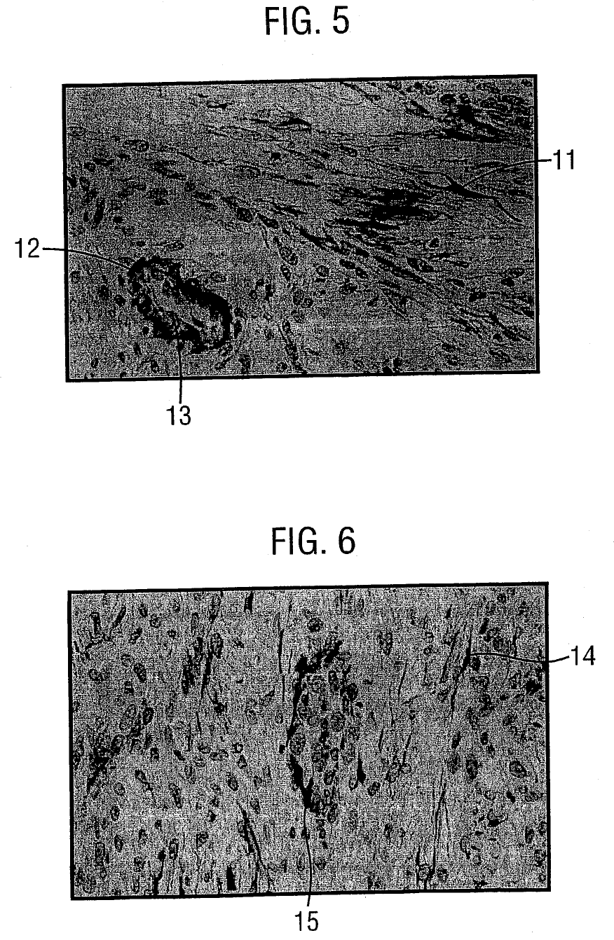

FIG. 5 is a photomicrograph of a paraffin section of skin at 60×magnification; and

FIG. 6 is a photomicrograph of a paraffin section of skin at 60×magnification.

Anti-human polyclonal antiserum was raised in rabbits and affinity purified against the LIP-1 [MERAGPSFGQQR] peptide n accordance with the method of Fawcett et al (Proc Natl Acod Sci USA 2000; 97:3702-3707), corresponding to amino acid residues 1-12 of human PDE5A1. LIP-1 is specific for PDE5 A1.

4 μm sections of formalin-fixed paraffin embedded tissue were cut and picked up on to APES (3-aminopropyltriethoxysilane) coated slides and dried at 60° C. for 1 hour. Sections were de-waxed and rehydrated followed by proteolytic antigen retrieval in 0.1% trypsin in 0.1% calcium chloride [pH7-6] at 37° C. for 8 minutes. Following a brief water wash, endogenous peroxidase activity was blocked by incubation in 9 ml H₂O₂ made up to 100 ml with distilled water for 10 minutes. Sections were washed in tap water then transferred to PBS. Excess buffer was removed from the slide and test sections were incubated in LIP-1 antibody diluted 1:600 in PBS for 1 hour at room temperature. Negative controls were included by omission of the primary antibody. Positive control tissue used was human corpus cavernosum. Immunodetection was carried out using DAKO Rabbit Envision TM system with 3-amino-9-ethylcarbazole (3AEC) as a substrate chromogen (red/brown staining).

FIG. 1 illustrates a section of reactive but non-inflamed skin at the edge of a skin wound. The positive staining of the smooth muscle cells within the media of the venules and negative fibroblasts indicates the expression of PDE5 in the healing wound. Hyperplastic but intact squamous epithelium 1 is negative. The underlying dermis contains mature scar tissue with small and large venules 2. Note the positive dark staining of the smooth muscle cells within the media of the venules (Original mag.×10).

FIG. 2 is a paraffin section taken from the border between a healing ulcer of 14 days (left) and intact epithelium (right). Again, the positive staining of the smooth muscle cells within the media of the venules (right) and the spindle cells (myofibroblasts) within the base of the ulcer (left) indicates PDE5 expression. Hyperplastic but intact squamous epithelium (right) and necrotic inflammatory exudate 3 is negative. Note the positive dark staining of the smooth muscle cells within the media of the venules 4 and of spindle cells within the base of the ulcer 5 (original mag.×20).

FIG. 3 is a paraffin section taken from the healed ulcer base where fascicles of young scar tissue have replaced normal dermal structures. Positive staining of some of the spindle cells (myofibroblasts) (8) and of some vascular structures is again indicative of PDE 5 expression. (Original mag×20).

FIG. 4 is a higher power view of the paraffin section of skin of FIG. 3. The section is taken from the healed ulcer base where fascicles of young scar tissue have replaced normal dermal structures. PDE 5 expression is illustrated by the positive staining of some of the spindle cells (myofibroblasts) (9) and of some of the microvessels which have thin media (10). (Original mag×40).

FIG. 5 is a higher powered view of FIG. 4 and shows a section taken from the healed ulcer base of FIG. 4 where fascicles of young scar tissue have replaced normal dermal structures. There is positive staining of some of the spindle cells (myofibroblasts) (11) which are present in acellular collagen. The immunolocalisation in the cytoplasm of some of these spindle cells has a patchy distribution. Positive staining of the medial smooth muscle cells within a small arteriole (12) indicates PDE 5 expression. There is negative staining of the lining endothelial cells (13) indicating the absence of PDE 5. (Original mag.×60).

FIG. 6 is also a higher powered view of FIG. 4 showing a section from the healed ulcer base in an area of relatively young scar tissue. Again, positive staining of some of the spindle cells (myofibroblasts) (14) and medial smooth muscle cells within the small arteriole (centre) (15) is indicative of PDE 5. In some of these spindle cells the immunolocalisation has a patchy distribution. (Original mag.×60).

The following formulation examples are illustrative only and are not intended to limit the scope of the invention. Active ingredient means a cGMP PDE5 inhibitor.

Formulation 1:

A tablet is prepared using the following ingredients:

Sildenafil citrate (50 mg) is blended with cellulose (microcrystalline), silicon dioxide, stearic acid (fumed) and the mixture is compressed to form tablets.

Formulation 2:

An intravenous formulation may be prepared by combining the active ingredient (100 mg) with isotonic saline (1000 ml).

Formulation 3:

A topical formulation may be prepared by combining up to 2% by weight of the active ingredient with a suitable excipient which may be a mixture with one or more of the following: mineral oil, liquid petrolatum, white petrolatum, propylene glycol, polyoxyethylene polyoxypropylene compound, emulsifying wax and water.

1. A method for reducing scarring and/or treating fibrosis in a patient which comprises treating the patient with an effective amount of a cGMP PDE5 inhibitor, or a pharmaceutical composition thereof.

2. A method as claimed in claim 1, wherein a disease associated with scarring and/or fibrosis is selected from: lung fibrosis, atherosclerosis, cardiovascular disease, dermal and corneal scarring and/or fibrosis following infection, trauma, surgery or thermal injury, scleroderma and other connective tissue disorders, fibrosis of the heart, chronic obstructive pulmonary disease, muscle fibrosis, kidney fibrosis, chronic dermal ulceration and lipdermatosclerosis, lung fibrosis or any origin), post-surgical and idiophatic adhesions, inflammatory conditions of the skin (including lichen and associated conditions), ageing and all ageing associated degenerative disorders (including ageing of the skin), liver fibrosis or any etiology (including viral and non-viral hepatitis, liver cirrhosis, chronic pancreatitis, chronic thyroiditis, calcinosis (of any origin), conditions whose pathogenesis is related to the deposition/remodelling of a connective matrix (including cancer).

3. The use of a cGMP PDE5 inhibitor for the manufacture of a medicament for reducing scarring and/or treating fibrosis.

4. Use as claimed in claim 3, wherein a disease associated with scarring and/or fibrosis is selected from: lung fibrosis, atherosclerosis, cardiovascular disease, dermal and corneal scarring and/or fibrosis following infection, trauma, surgery or thermal injury, scleroderma and other connective tissue disorders, fibrosis of the heart, chronic obstructive pulmonary disease, muscle fibrosis, kidney fibrosis, chronic dermal ulceration and lipdermatosclerosis, lung fibrosis or any origin), post-surgical and idiophatic adhesions, inflammatory conditions of the skin (including lichen and associated conditions), ageing and all ageing associated degenerative disorders (including ageing of the skin), liver fibrosis or any etiology (including viral and non-viral hepatitis, liver cirrhosis, chronic pancreatitis, chronic thyroiditis, calcinosis (of any origin), conditions whose pathogenesis is related to the deposition/remodelling of a connective matrix (including cancer).

5. A method or use as claimed in any of claims 1 to 4, wherein the inhibitor is administered orally or topically.

6. A method or use as claimed in any preceding claim, wherein the wherein the inhibitor has an IC50 at less than 100 nanomolar.

7. A method or use as claimed in claim 6, wherein the inhibitor has a selectivity ratio in excess of 1000.

8. A method or use as claimed in any preceding claim, wherein the inhibitor is sildenafil.

9. A method or use as claimed in claim 8, wherein the daily dosage is 5 to 500 mg.

10. A method or use as claimed in claim 9, wherein the daily dosage is 10 to 100 mg.

11. The use of a cGMP PDE5 inhibitor in combination with a PCP and/or PDE4 inhibitor for the manufacture of a medicament for reducing scarring and/or treating fibrosis.

12. A pharmaceutical pack comprising: a pharmaceutical composition comprising a PDE5 inhibitor, directions relating to the use of the composition for reducing scarring and/or treating fibrosis, and a container.

13. A combination of a PDE5 inhibitor together with a PCP inhibitor and/or a PDE4 inhibitor (uPA).
